(19) United States
(12) Patent Application Publication
(10) Pub. No.: US 2008/0305990 A1
(43) Pub. Date: Dec. 11, 2008

(54) Method of Producing Fully Carbamylated Erythropoietin

(75) Inventors: Michael Brines, Woodbridge, CT (US); Anthony Cerami, Somers, NY (US); Carla Hand, Chapel Hill, NC (US); Qiao-wen Xie, Yonkers, NY (US)

Correspondence Address: JONES DAY, 222 EAST 41ST ST, NEW YORK, NY, 10017, US

(73) Assignee: The Kenneth S Warren Institute, Inc., Ossining, NY (US)

(21) Appl. No.: 11/631,458

(22) PCT Filed: Jul. 1, 2005
(86) PCT No.: PCT/US05/23505

§ 371 Date: Jan. 3, 2007

Related U.S. Application Data
(60) Provisional application No. 60/585,262, filed on Jul. 2, 2004.
(60) Provisional application No. 60/584,951, filed on Jul. 2, 2004.

(51) Int. Cl. A61K 38/22; C07K 14/575; A61P 1/16; A61P 9/00; A61P 27/02

(52) U.S. Cl. 514/8; 530/397

(57) ABSTRACT

The present invention relates to a method of carbamylating an erythropoietin such that the resulting carbamylated erythropoietin has less that about 10% free primary amines on the lysines and the N-terminal amino acids, is not digested when exposed to Lys-C proteolysis, exhibits no erythropoietic activity in a TF-1 or UT-7/EPOR cell viability assay at a concentration of 1 μg/ml, and demonstrates a static sciatic index of less than about 0.65 within a Sciatic Nerve Assay. Additionally, the invention is related to pharmaceutical compositions containing carbamylated erythropoietins of the invention and the use of the pharmaceutical compositions for the treatment of conditions and diseases of excitable tissues.

### BACKGROUND OF THE INVENTION

Recently it has been discovered that erythropoietin possesses tissue protective activity in addition to its previously recognized hematopoietic activities. PCT/US00/10019. Further studies into the tissue protective aspects of erythropoietin have indicated that the two activities can be separated out, and that this may be accomplished by various modifications, such as chemical and mutational modifications, to the amino acid backbone of erythropoietin. PCT/US01/49479 and PCT/US03/20964. In particular, it has been noted that a tissue protective cytokine can be made by carbamylating one or more of the primary amino groups of erythropoietin, among the lysines or the N-terminal amino acid. PCT/US01/49479.

The carbamylation of protein amino groups will occur naturally in the presence of urea. This is due to isocyanic acid, the reactive form of ammonium cyanate in equilibrium with urea, reacting with primary amino groups on the N-terminal amino acids as well as the side chains of lysines. This carbamylation has been observed within erythropoietin as well.

Methods of carbamylating proteins have been disclosed as well. G R Stark, Methods in Enzymology 11, 590-594 (1967), G R Stark, W. H. Stein, and S. Moore, J. Biol. Chem. 235, 3177-3181 (1960). Additionally, methods have been described for selectively carbamylating one amino group over another, i.e preferential carbamylation of lysines. Zeng, J. (1991) Lysine modification of metallothionein by carbamylation and guanidination. Methods in Enzymology, 205:433-437. The carbamylation of erythropoietin has been evaluated to determine its detrimental effects upon its erythropoietic activity. K. C. Mun and T. A. Golper, (2000) Impaired biological activity of erythropoietin by cyanate carbamylation. Blood Purif. 18, 13-17; R. Satake, H. Kozutsumi, M. Takeuchi, K. Asano, (1990) Chemical modification of erythropoietin: an increase in in vitro activity by guanidation. Biochim. Biophys Acta 1038, 125-129; and L. O. Pedersen et al., Eur J Immunol 25, 1609-1616 (1995). However, these earlier studies merely evaluated the effects of carbamylation of the lysines of erythropoietin solely as it pertains to the hematopoietic effects of erythropoietin without any recognition of whether the carbamylated erythropoietin retained any tissue protective activity. Additionally, these articles characterized the carbamylation in terms of its effects upon erythropoiesis as opposed to the actual extent of carbamylation of amino acids that occurred within erythropoietin.

Furthermore, given the newly discovered therapeutic uses of carbamylated erythropoietin, a need exists for an assay to confirm the therapeutic activity of carbamylated erythropoietin especially as a release assay for purposes of manufacturing it in accordance with regulatory requirements. Several assays have been disclosed to assess the tissue protective effect of compounds for example, traumatic brain injury, traumatic spinal cord injury, stroke models, EAE models for multiple sclerosis as disclosed within PCT/US01/49479, PCT/US03/20964, PCT/US03/21350, PCT/US04/15733, PCT/US04/15863, and U.S. application Ser. No. 10/185,841 hereby incorporated by reference. However, these assays require substantial amounts of time and skilled personnel to complete, and validation of the tissue protective effects of the compound may not occur for several weeks or months following initiation of the assay. Preferably, a release assay should be able to be completed within less time and provide highly reproducible results. Thus, a need still exists for a quick and reproducible assay for validating the therapeutic activity of carbamylated erythropoietin.

There remains a need for a method to produce carbamylated erythropoietin that exhibits a consistent level of carbamylation without undesirable levels of contaminants such as aggregates. In light of the regulatory requirements of the Food and Drug Administration that a biologic compound be well characterized and consistent, a need exists for a method of confirming the characteristics of a carbamylated erythropoietin and a biological release assay to readily verify the activity of the biological compound.

### BRIEF SUMMARY OF THE INVENTION

The present invention relates to a method for producing a carbamylated erythropoietin having less that about 10% free primary amines on the lysines and the N-terminal amino acids. The method involves contacting an amount of erythropoietin at a concentration of less than 4 mg/ml, with a concentration of about 0.05 M to 2 M potassium cyanate, with a concentration of about 0.05 M to 0.5 M sodium borate buffer pH 7-10, at a temperature of about 30 to 38° C. for a period of about 1 to 24 hours. The resulting carbamylated erythropoietin is not digested when exposed to Lys-C proteolysis, exhibits no erythropoietic activity in a TF-1 or UT-7/EPOR cell viability assay at a concentration of 1 μg/ml, and demonstrates a static sciatic index of less than about 0.65 within a Sciatic Nerve Assay. Most preferably, only the primary amino groups of lysine and N-terminal amino acids are carbamylated.

In a preferred embodiment, the carbamylated erythropoietin of the method has less than about 7.5% free primary amines on the lysines and the N-terminal amino acids, and in the most preferred embodiment the carbamylated erythropoietin has less than about 5% free primary amines on the lysines and the N-terminal amino acids. In another embodiment, the carbamylated erythropoietin has less than 10% aggregates, in a preferred embodiment it has less than 6% aggregates, and in the most preferred embodiment it has less than 2% aggregates.

In an embodiment of the current method, the erythropoietin is recombinant erythropoietin, long acting erythropoietin, erythropoietin derivatives, erythropoietin analogs, erythropoietin conjugates, erythropoietin fusion proteins, chemically modified erythropoietin, erythropoietin muteins, expression-system-mediated glycosylation modifications of erythropoietin, synthetic erythropoietin, or naturally occurring erythropoietin. In a preferred embodiment, the erythropoietin is human erythropoietin. In another preferred embodiment, the erythropoietin is asialoerythropoietin.

Also, in a preferred embodiment of the method the concentration of erythropoietin in the reaction is about 1.1 mg/ml to about 2.5 mg/ml and more preferably about 2.2 mg/ml. The potassium cyanate of the present method is present in the reaction in a concentration of about 0.5 M to about 1.5 M, most preferably at about 1 M. Also, in a preferred embodiment of the method sodium borate buffer is present in the reaction in a concentration of about 0.1 M to about 0.5 M and more preferably at a concentration of about 0.5 M. Additionally, the pH of the buffer is preferably 8.7-9.2 pH.

Preferably the reaction is conducted at a temperature of about 36° C. to about 38° C. In the most preferred embodiment of the method the temperature is about 37° C. The reaction, in a preferred embodiment, is conducted for about 14 to 24 hours, and in the most preferred embodiment for about 16 hours.

In a preferred embodiment of the method, the carbamylated erythropoietin exhibits no erythropoietic activity in a TF-1 or UT-7/EPOR assay at a concentration of 10 μg/ml. In another preferred embodiment, the static sciatic index for the carbamylated erythropoietin is less than about 0.62, and in the most preferred embodiment the static sciatic index for carbamylated erythropoietin is less than about 0.60.

The current invention also relates to a pharmaceutical composition comprising a therapeutically effective amount of a carbamylated erythropoietin wherein the carbamylated erythropoietin has less than about 10% free primary amines on the lysines and the N-terminal amino acids is not digested when exposed to Lys-C proteolysis, exhibits no erythropoietic activity in a TF-1 or UT-7/EPOR cell viability assay at a concentration of 1 μg/ml, and demonstrates a static sciatic index of less than about 0.65 within a Sciatic Nerve Assay, and a pharmaceutically acceptable carrier.

In a preferred embodiment of the pharmaceutical composition, the carbamylated erythropoietin has less that about 7.5% free primary amines on the lysines and the N-terminal amino acids, and in a most preferred embodiment, the carbamylated erythropoietin has less that about 5% free primary amines on the lysines and the N-terminal amino acids.

Also, in a preferred embodiment of the invention, the carbamylated erythropoietin in the pharmaceutical composition exhibits no erythropoietic activity in a TF-1 or UT-7/EPOR cell viability assay at a concentration of 10 μg/ml. The pharmaceutical composition in another embodiment has a carbamylated erythropoietin with a static sciatic index is less than 0.62, and preferably less than 0.60.

The present invention also relates to a method for treating a condition or disease of an excitable tissue comprising administering a non-toxic amount of the pharmaceutical composition. In one embodiment, the excitable tissues treatable are the heart, eye or renal tissue. In another embodiment, the conditions or diseases being treated are optic neuritis, blunt or penetrating injuries to the eye, infections of the eye, sarcoid, sickle cell disease, retinal detachment, temporal arteritis, retinal ischemia, macular degeneration, retinal detachment, retinitis pigmentosa, arteriosclerotic retinopathy, hypertensive retinopathy, retinal artery blockage, retinal vein blockage, hypotension, diabetic retinopathy, diabetic neuropathy, coronary artery disease, myocardial infarction, Dressler's syndrome, angina, congenital heart disease, valvular cardiomyopathy, prinzmetal angina, cardiac rupture, aneurysmatic septal perforation, angiitis, arrhythmia, congestive heart failure, cardiomyopathies, myocarditis, cor pulmonale, blunt or penetrating traumas to the heart, toxic poisoning, renal failure, vascular/ischemic, interstitial disease, diabetic kidney-disease, nephrotic syndromes, kidney infections, or Henoch Schönlein purpura.

### BRIEF DESCRIPTION OF THE FIGURES

FIG. 1 shows the UV absorbance of a carbamylated erythropoietin manufactured in accordance with the current method as detailed in Example 1.

FIG. 2 shows the results of an isoelectric focusing (IEF) gel of a carbamylated erythropoietin manufactured in accordance with the method of Example 1.

FIG. 3 shows the SDS-PAGE analysis of a carbamylated erythropoietin manufactured in accordance with the method of Example 1 which demonstrates the absence of aggregates.

FIG. 4 shows the size exclusion (SE)-HPLC analysis of a carbamylated erythropoietin manufactured in accordance with the method of Example 1 which confirms the absence of aggregates.

FIG. 5 shows the results of a 16% tricine gel of a deglycosylated carbamylated erythropoietin in accordance with Example 1 demonstrating that the carbamylation of the lysines was complete.

FIG. 6 shows the results of a UT-7 assay of the carbamylated erythropoietin from Example 1 demonstrating the compounds lack erythropoietic activity.

FIG. 7 illustrates the Toe Spread and Intermediate Toe Spreads in rats treated with carbamylated erythropoietin and saline in a Sciatic Nerve Assay.

FIG. 8 shows the results of a Sciatic Nerve Assay of the carbamylated erythropoietin from Example 1 demonstrating that the carbamylated erythropoietin has tissue protective activity.

FIG. 9 shows the UV absorbance of an erythropoietin in a TNBS assay.

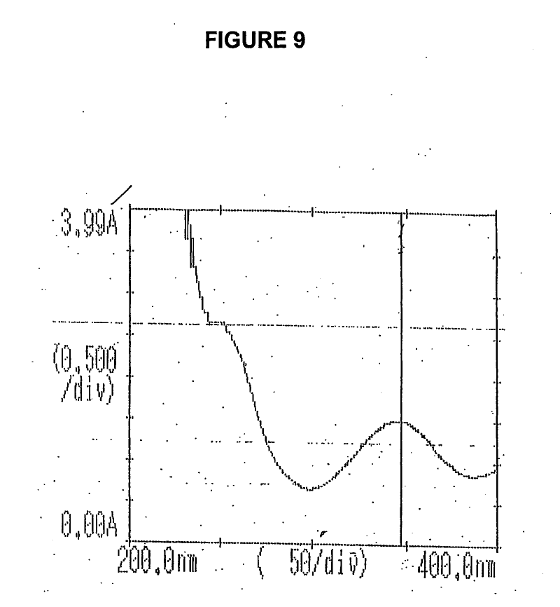

FIG. 10 shows the UV absorbance of a blank in a TNBS assay.

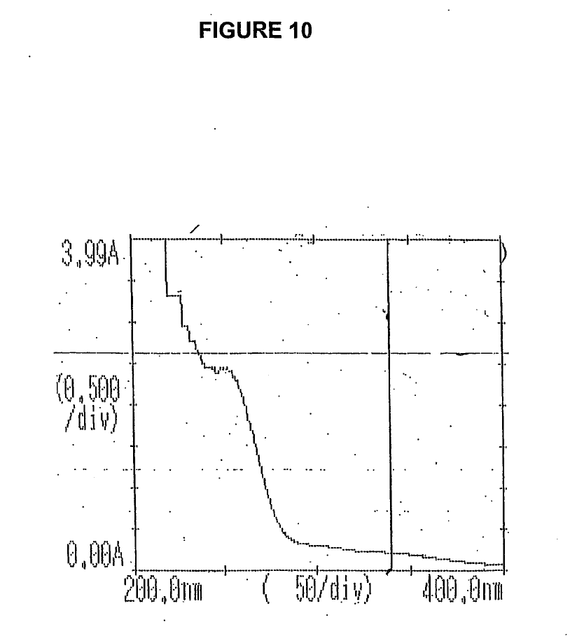

FIG. 11 shows the UV absorbance of a carbamylated erythropoietin manufactured in accordance with the current method as detailed in Example 1 within a TNBS assay.

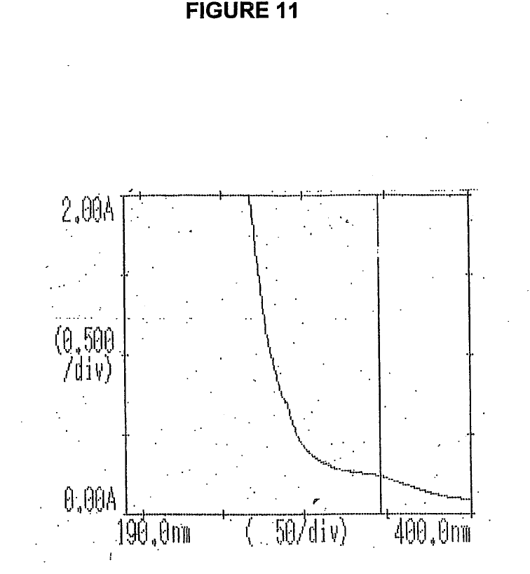

### DETAILED DESCRIPTION OF THE INVENTION

The carbamylation process of the present invention provides for the selective carbamylation of the primary amines of the eight lysines and the N-terminal amino acid in erythropoietin. In a preferred embodiment the process results in the exclusive carbamylation of the primary amines of the lysines and the N-terminal amino acid, herein referred to as fully carbamylated erythropoietin. Essentially the process consists of the following steps:
A) Concentration of erythropoietin.
B) Carbamylation of erythropoietin.
C) Desalting.
D) Purification of Fully Carbamylated Erythropoietin.
E) Analysis of Fully Carbamylated Erythropoietin.
F) Verification of non-erythropoietic activity and tissue protective activity using in vitro and in vivo assays.

### A. Concentration of Erythropoietin.

Erythropoietin is a glycoprotein hormone which in humans has a molecular weight of about 34 kDa. The mature protein comprises about 165 amino acids, and the glycosyl residues comprise about 40% of the weight of the molecule. The mature erythropoietin protein has eight lysine residues. These, in addition to the N-terminal amino acid (alanine), provide nine primary amino groups for potential carbamylation. Erythropoietin can be obtained commercially, for example, under the trademarks of PROCRIT, available from Ortho Biotech Inc., Raritan, N.J., EPOGEN, available from Amgen, Inc., Thousand Oaks, Calif., and RECORMON, available from Roche, Basel, Switzerland. In addition to native erythropoietins, other forms of erythropoietin useful in the practice of the present invention encompass chemical modifications, muteins and/or expression-system-mediated glycosylation modifications of naturally occurring, synthetic and recombinant forms of human and other mammalian erythropoietins. Various modified forms of erythropoietin have been described with activities directed towards improving the erythropoietic activity of the molecule, such as those with altered amino acids at the carboxy terminus described in U.S. Pat. No. 5,457,089 and in U.S. Pat. No. 4,835,260; erythropoietin isoforms with various numbers of sialic acid residues per molecule, such as described in U.S. Pat. No. 5,856,298; polypeptides described in U.S. Pat. No. 4,703,008; agonists described in U.S. Pat. No. 5,767,078; peptides which bind to the erythropoietin receptor as described in U.S. Pat. Nos. 5,773,569 and 5,830,851; small-molecule mimetics as described in U.S. Pat. No. 5,835,382; and chemically modified erythropoietins (for example asialoerythropoietin) or recombinant erythropoietins (for example, S100E or S100E/K97A erythropoietin muteins) lacking erythropoietic activity as described in PCT/US00/10019 and PCT/US03/20964. Additionally, modified forms of erythropoietin having an in vivo half life greater than that of either naturally occurring or recombinant human erythropoietin have been developed through the addition of sialic acid residues, glycosylation sites, polyethylene glycol (PEG), or portions of other proteins (fusion proteins) or any combination of the above. Examples of such long acting erythropoietins are ARANESP available from Amgen Inc., Thousand Oaks, Calif., CERA available from Roche, Basel, Switzerland, and the diglycosylated and pegylated erythropoietins taught in WO03029291. Long acting erythropoietins include, but are not limited to, erythropoietins having an extended half life due to increased sialic acid residues as taught in U.S. Pat. No. 5,856,298, the addition of sugars as taught in EP0640619, the addition of polyethylene glycol (PEG) residues as taught in WO0102017 and WO0032772, the addition of proteins through fusion with erythropoietin as taught in U.S. Patent Application Serial Nos. 20040009902, 20030124115, and 20030113871 as well as U.S. Pat. No. 6,242,570, chemical modifications, the modification of the naturally occurring glycosylation pattern of either recombinant or naturally occurring human erythropoietin as taught in PCT application number US94/02957 and U.S. Patent Application Serial No. 20030077753, and/or mutations as taught in U.S. Patent Application Serial No. 20020081734. Additional long acting erythropoietins include diglycosylated and pegylated erythropoietin conjugates taught in the following patent applications WO0102017, EP1064951, EP1345628, WO03029291, EP0640619, US2003077753, US20030120045 and U.S. Pat. Nos. 6,583,272 and 6,340,742. For purposes of the present invention, reference to erythropoietin shall include erythropoietin, long acting erythropoietin, erythropoietin derivatives, erythropoietin analogs, erythropoietin conjugates, erythropoietin fusion proteins, and the like.

Although the process can be performed with erythropoietin in solution, it is best for the speed and completeness of the reaction to have a low process volume of the solution. The erythropoietin may be concentrated using ultrafiltration methods including, but not limited to, centrifugal filtration and stirring filtration. A molecular weight cut-off (MWCO) membrane of equal to or less than about 10 KDa is used for the ultrafiltration process. After the concentration procedure, erythropoietin should be present at a concentration of greater than about 2 mg/ml to less than or equal to about 20 mg/ml, preferably about 2.2 to about 10 mg/ml, most preferably about 4 mg/ml to 6 mg/ml.

### B. Carbamylation of Erythropoietin.

After the erythropoietin is concentrated, carbamylation of the erythropoietin is performed. The reagents for the reaction consist of cyanate and buffer in addition to the erythropoietin. Several factors affect the carbamylation procedure including, but not limited to, (1) concentrations of reagents (erythropoietin, cyanate); (2) buffer and pH of the reaction, (3) temperature of reaction, and (4) length of time of reaction. These are discussed below.

### (1) Concentration of Reagents.

(a) Erythropoietin.

In the carbamylation reaction solution the concentration of erythropoietin will be about half the above noted concentrations, i.e. about 1 mg/ml to less than or equal to about 10 mg/ml, preferably about 2 mg/ml to 4 mg/ml, and most preferably about 2 mg/ml to 3 mg/ml.

(b) Cyanate.

Appropriate cyanates for the present process include, but are not limited to, potassium cyanate, sodium cyanate, ammonium cyanate or any other acceptable cations. Preferably, the cyanate is a potassium cyanate. Also, prior to carbamylation, the cyanate is preferably recrystallized from ethanol (50-100%). Additionally, in order to verify the potency of the recrystallized cyanate, a small pilot carbamylation reaction may be performed to verify that the erythropoietin becomes fully carbamylated with the recrystallized cyanate as used. The concentration of cyanate within the reaction solution is preferably about 0.05 M to 1.75 M, more preferably about 0.5 M to 1.5 M, and most preferably 1 M.

### (2) Buffer and pH of the Reaction.

Preferably, the buffer should be able to maintain the pH of the solution at about 7-10 and most preferably about 8.7-9.2. Suitable buffers include any amine free buffers including, but not limited to, phosphate buffers and borate buffers. Preferably the buffer is a borate buffer, more preferably it is a sodium borate buffer. The concentration of buffer within the carbamylation reaction solution is preferably about 0.05 M to 0.5 M, and most preferably about 0.5 M.

### (3) Temperature of the Reaction.

The reaction solution is maintained at a suitable temperature. In particular, the temperature of the solution may be maintained at a temperature of 30-38° C., preferably about 36-38° C., most preferably about 37° C.

### (4) Time of the Reaction.

The reaction should be conducted for a time sufficient to result in the carbamylation of all of the lysines and the N-terminal amino group of the erythropoietin. The reaction may be conducted for about 1 hour to about 24 hours, preferably about 6 hours to about 24 hours, more preferably about 14 hours to about 17 hours, and most preferably about 16 hours.

### C. Desalting.

Subsequent to the carbamylation reaction, the reaction solution is desalted. This may be accomplished by various methods, including but not limited to, dialysis, desalting column, or centrifugal filter device. For example, the reaction solution can be dialyzed (with multiple changes) against about 100- to 1000-fold volume of distilled water, phosphate buffer (pH ˜7.2), citrate buffer (pH ˜6.8), or 10 mM Tris-HCl buffer (pH 8.6) at about 2 to 8° C. Alternatively, a PD-10 column with G-25 Sephadex (both available from Amersham Biosciences Corp., Piscataway, N.J.) may be used to perform the desalting.

### D. Purification.

After desalting, the reaction solution is purified to isolate the carbamylated erythropoietin and remove aggregates. The purification of the reaction solution may be accomplished using various chromatography methods, including but not limited to affinity chromatography, ion exchange chromatography, hydrophobic interaction chromatography, gel filtration (size exclusion) chromatography, reverse phase chromatography and ultrafiltration techniques. The purification may be accomplished using any one of the above noted methods or a combination of those methods, see e.g. Protein Purification Handbook, 18-1132-29, Amersham Pharmacia Biotech. For example, purification of the carbamylated erythropoietin may be accomplished using a gel filtration column, such as Sephacryl S-100, with a 50 mM Na-phosphate buffer with 0.15 M NaCl at a pH of about 7.0-7.2.

The result of this final procedure is a carbamylated erythropoietin having less than about 10% free primary amines (i.e. greater than about 90% of the lysines modified to homocitrulline), preferably less than about 7.5% free primary amines (i.e. greater than about 92.5% of the lysines modified to homocitrulline), and most preferably less than about 5% free primary amines (i.e. greater than about 95% of the lysines have been modified to homocitrulline). Additionally, the carbamylated erythropoietin should have less than about 10% aggregates within the solution, preferably less than about 6% aggregates, most preferably less than about 2% aggregates.

### E. Analysis of Carbamylated Erythropoietin

Upon completion of the carbamylation procedure it is necessary to confirm: (1) the erythropoietin is completely carbamylated; (2) the carbamylated erythropoietin is pure and without aggregates; and (3) the carbamylated erythropoietin lacks erythropoietic activity and (4) the carbamylated erythropoietin is tissue protective.

### (1) Completeness of Carbamylation.

The complete carbamylation of the lysines and N-terminal amino groups may be verified using several techniques, including, but not limited to, proteolysis of the carbamylated erythropoietin (using Lys C digestion, tryptic digestion, acid or alkaline hydrolysis etc.) followed by mass spectrometry (LC/MS/MS), matrix assisted laser desorption ionisation (MALDI-TOF), MALDI TOF/TOF®, electrospray ionisation (ESI-TOF), triple quadrupole TOF, and ESI-MS/MS), gel electrophoresis or isoelectric focusing gel electrophoresis (IEF), or amino acid analysis (for homocitrulline).

For example, an IEF gel can be used initially to confirm that carbamylation occurred successfully. When the carbamylation is successful, the IEF gel of the carbamylated erythropoietin will show a pI of less than 3.5 in comparison to erythropoietin which will have a pI of about 3.5 to 5.

A more exact measure of the extent of carbamylation of erythropoietin can be determined using analysis of Lys-C digests of the carbamylated erythropoietin by PAGE such as on a 16% tricine gel or 18% tri-glycine gel. Lys-C is an endopeptidase which cuts the protein after unmodified lysine residues (if not followed by an acidic amino acid). There are eight (8) lysine residues in the erythropoietin molecule but two (2) of them are followed by glutamic acids. Thus, Lys-C cuts erythropoietin at six sites into seven (7) smaller peptides, which migrate faster than the non-digested carbamylated erythropoietin. When all six of the lysine residues are carbamylated, the resulting carbamylated erythropoietin will not be digested by Lys-C and the Lys-C treated carbamylated erythropoietin will migrate to the same spot as the carbamylated erythropoietin that has not been digested by Lys-C. When a carbamylated erythropoietin product is not completely carbamylated, it would be partially digested by Lys-C. Therefore, the gel analysis of the Lys-C digests of a carbamylated erythropoietin product provides an estimate of the level of carbamylation. Preferably, the Lys-C digestion may be performed with prior deglycosylation of the carbamylated erythropoietin using PNGase.

For Lys-C digestion, samples (200 μg) are dried under vacuum and dissolved in 200 μl 6 M guanidinium-HCl, 250 mM Tris pH 9.5. Twenty-five μl of 0.1 M dithioerythritol (DTE) is added and the incubation continued in the dark at 37° C. After 30 min, 25 μl of iodoacetamide (IAA) (0.6 M) is added and the incubation is continued for 60 min at room temperature in the dark. Finally, the sample is desalted on a 5 ml HiTrap G025 column (Amersham-Biosciences, Little Chalfont, UK) into 50 mM NH₄HCO₃, 0.4 M urea pH 8.3. One ¼ volume (˜50 μg protein) is incubated with 2 μg of Lys-C proteinase of Achromobacter lyticus (Roche, Mannheim, Germany) for 20 h at 37° C. Digested samples are either analyzed by RP-HPLC or by SDS-PAGE (NuPAGE 4-12% using MES buffer system, Invitrogen, Carlsbad, Calif.).

Additionally, a Trinitrobenzenesulfonic Acid (TNBS) Assay can be used to measure the free amino groups (lysines and N-terminal amino acid) remaining within the fully carbamylated erythropoietin. In this assay, three assays are run, one for erythropoietin, one for buffer (control) and one with the carbamylated erythropoietin. Each sample is mixed with TNBS in borate buffer (0.3 M, pH>9.5) in a dark colored tube to achieve a final concentration of 0.5 mg/ml for protein, 0.3 mM for TNBS and the total reaction volume of 0.5 to 1.0 ml. The mixture is permitted to react for 1 hour at room temperature and is then transferred to a microcuvette. The cuvette is then scanned at 200-400 nm in a spectrophotomer. The scanning results are printed out for each sample and the peak and the peak absorbance are identified for each sample. The percentage of free amino groups within the carbamylated erythropoietin is then computed as follows: (peak absorbance for carbamylated erythropoietin sample-peak absorbance for the blank)/peak absorbance for erythropoietin. For purposes of this evaluation, erythropoietin is assumed to have 100% free amino groups. The percentage of free amino groups within a fully carbamylated erythropoietin is below about 10%, preferably the percentage of free amino groups within a fully carbamylated erythropoietin is below 7.5%, most preferably the percentage of free amino groups within a fully carbamylated erythropoietin is below 5%.

Additionally, amino acid mapping (for homocitrulline) and mass spectrometry (such as MALDI-TOF and LC/MS), may be used to determine that the primary amines of all eight lysines and the N-terminal amino acid were carbamylated. Furthermore, these methods may be used to analyze the protein and confirm that only the primary amines of the lysines and N-terminal amino acid are carbamylated in the fully carbamylated erythropoietin.

### (2) Purity and Removal of Aggregates.

According to the method of the present invention, the absence/low level of aggregates and protein content in the carbamylated erythropoietin product are confirmed. The removal of aggregates can be confirmed using electrophoresis such as sodium dodecylsulfate-Polyacrylamide Gel Electrophoresis (SDS-PAGE, under reducing and non-reducing conditions) and liquid chromatography such as SE-HPLC analysis. Additionally, UV scanning (A280) or Enzyme-Linked Immunosorbent Assay (ELISA) can be used to confirm the protein content.

### (3) Verification of Carbamylated Erythropoietin Activity.

### (a) Lack of Hematopoietic or Erythropoietic Activity.

The non-erythropoietic activity of a recombinant tissue protective cytokine modified or as described herein can be verified using TF-1 or UT-7/EPOR in vitro assays. In the TF-1 assay, TF-1 cells, a human erythroleukemia cell line (available from ATCC), are grown in a complete RPMI-1640 medium (10% FCS) supplemented with 5 ng/ml of GM-CSF at 37° C. in a CO₂ incubator. On day one the cells are washed twice in and suspended in starvation medium (5% FCS without GM-CSF) at a density of 10⁶ cells/ml followed by incubation for 16 hours. On day 2, a 96 well plate is prepared by: (1) adding 100 μl of sterile water to the outer wells to maintain moisture; (2) adding starvation medium (5% FCS without cells or GM-CSF) alone to 5 wells as blanks; (3) seeding 25,000 cells/well in 5 wells as cell control without reagent, (4) seeding 25,000 cells/well with escalating concentrations of erythropoietin (5 wells per concentration of erythropoietin) and (5) seeding 25,000 cells/well with escalating concentrations of the carbamylated erythropoietin sample in the remaining wells (five wells per concentration of carbamylated erythropoietin). The contents are mixed briefly and carefully, using the orbital vibrating platform seated on top of the stir plate. The different concentrations of erythropoietin and carbamylated erythropoietin used within the assay are from 0.1 ng/ml to 10 μg/ml. The 96 well plate is then incubated for 48 h in a humidified incubator with 5% CO₂ at 37° C. On day four of the assay, a solution of 15 μl WST-1 Cell Proliferation Reagent (Roche) is added to each well, incubated for 1 hour at 37° C. in CO₂. After mixing 1 minute, read the plate in a plate reader (absorption at 450 nm, subtracted from background absorption at 650 nm). This procedure measures the formazan product formed during cellular metabolism of the tetrazolium dye, which correlates with cellular viability/number of cells. If the cells fail to proliferate at a concentration equal in the presence of 1 μg/ml and preferably 10 μg/ml carbamylated erythropoietin, the non-erythropoietic activity of the carbamylated erythropoietin has been confirmed.

Additionally, a human erythropoietin-dependent leukemia cell line, UT-7/EPOR, is used for the determination of the erythroid effect of the carbamylated erythropoietin. UT-7/EPOR cells (Deutsche Sammlung von Mikroorganismen und Zellkulturen (DSMZ), Cat. No. ACC 363) are normally grown in a complete RPMI-1640 medium with (10% FBS) supplemented with 5 ng/ml erythropoietin. The proliferation/survival (=viability increase) response of the cells exposed to erythropoietin is mediated by the homodimeric classical erythropoietin receptor. The proliferation response is a quantitative measure of and correlates with the capacity of erythropoietin variants to stimulate the classical erythropoietin receptor. The UT-7/EPOR assay, which is similar to the TF-1 assay disclosed above, is performed by transferring the cells to fresh complete RPMI 1640 medium supplemented with erythropoictin (5 ng/ml). The cells are then grown in the 75 cm² flasks with 20 ml of culture/flask. On day two of the assay the cells are washed two times and are re-suspended in starvation media (containing 3% serum instead of 10%) at a density of 4×10⁵ cells/ml in a 25 cm² flask. The cells are then incubated for 4 h in a humidified incubator with 5% CO₂ at 37° C. At the end of the 4-hour incubation, a 96 well plate is prepared and the remainder of the procedure is the same as the TF-1 assay noted above with the exception of seeding 20,000 cells per well. Preferably, in both assays, the carbamylated erythropoietin will have no erythropoietic activity for a dose lower than 1 μg/ml, and more preferably for a dose lower than 10 μg/ml.

### (b) Tissue Protective Activity.

Additionally, the present invention relates to a robust, efficient and effective release assay for confirming the tissue protective activity of erythropoietin. Specifically, the current invention utilizes a Sciatic Nerve Assay as a release.

The Sciatic Nerve Assay is performed using Sprague-Dawley rats. Under isoflurane anesthesia, the rat's core temperature is controlled at 37° C. by a thermal blanket and the operating room's temperature is maintained above 23° C., and the left sciatic nerve of the rat is exposed at mid-thigh. A ligature of 2-0 silk (Ethicon 685G) is placed around the sciatic nerve, stabilized with a rigid polyethylene tube and a 100 g weight attached via a pulley system to apply traction for one minute. A single dose of carbamylated erythropoietin or control (saline or a bovine serum albumin solution at the same concentration as tissue protective cytokine) is administered i.v. immediately following release of the ligature, and the animals maintained on a heating blanket until fully recovered. Neurological function was scored by analyzing the footprints in triplicate of rats standing on a digital scanner (S. Erbayraktar et al., Proc Natl Acad Sci USA 100, 6741-6746 (2003); G. Grasso et al. Med Sci Monit, 2004; 10(1):BR1-3). Parameters were compared for injured (left) vs uninjured (right) sides to obtain the sciatic static index (SSI; ibid). Analysis was carried out every day after surgery for 4 consecutive days, and the area under the curve is calculated to score the animals. The SSI for the rats treated with the carbamylated erythropoietin will be less than the SSI for the PBS treated rat if the carbamylated erythropoietin is tissue protective. Preferably the SSI for the carbamylated erythropoietin will be below 0.65, more preferably the SSI for the carbamylated erythropoietin will be below 0.62, and most preferably below 0.60.

Under the above conditions the Sciatic Nerve Assay of the current invention has demonstrated a reproducible level of injury and consistent response, and therefore this assay provides a robust method to validate the tissue protective effects of the carbamylated erythropoietin within five days of initiating the assay. Given the relatively quick readout of this assay and its robustness of this assay it also provides a practical and convenient mechanism for assessing dose ranges, methods of administration and other pharmokinteic attributes of the compound.

### F. Further Modification

Once the attributes of the fully carbamylated erythropoietin ((1) the erythropoietin is completely carbamylated; (2) the carbamylated erythropoietin is pure and without aggregates; and (3) the carbamylated erythropoietin lacks erythropoietic activity and (4) the carbamylated erythropoietin is tissue protective) have been confirmed, the fully carbamylated erythropoietin may be subjected to further modification. Such modifications may include, but are not limited to, deglycosylation, pegylation, fusion with other proteins, and additional chemical modifications.

Also, the fully carbamylated erythropoietin of the present invention may be further modified by associating it with another molecule for the purpose of facilitating the transport of the molecule across an endothelial cell barrier in a mammal. Tight junctions between endothelial cells in certain organs in the body create a barrier to the entry of certain molecules. For treatment of various conditions within the barriered organ, means for facilitating passage of pharmaceutical agents is desired. Carbamylated erythropoietin, including the fully carbamylated erythropoietin of the current invention, is useful as a carrier for delivering other molecules across the blood-brain and other similar barriers. A composition comprising a molecule desirous of crossing the barrier with carbamylated erythropoietin is prepared and peripheral administration of the composition results in the transcytosis of the composition across the barrier. The association between the molecule to be transported across the barrier and the carbamylated erythropoietin may be a labile covalent bond, in which case the molecule is released from association with the carbamylated erythropoietin after crossing the barrier. If the desired pharmacological activity of the molecule is maintained or unaffected by association with carbamylated erythropoietin such a complex can be administered.

The skilled artisan will be aware of various means for associating molecules with fully carbamylated erythropoietin of the invention and the other agents described above, by covalent, non-covalent, and other means. Furthermore, evaluation of the efficacy of the composition can be readily determined in an experimental system. Association of molecules with carbamylated erythropoietin may be achieved by any number of means, including labile, covalent binding, cross-linking, etc. Biotin/avidin interactions may be employed; for example, the carbamylated erythropoietin may be biotinylated and then complexed with a labile conjugate of avidin and a molecule desirably transported. As mentioned above, a hybrid molecule may be prepared by recombinant or synthetic means, for example, a fusion or chimeric polypeptide which includes both the domain of the molecule with desired pharmacological activity and the domain responsible for tissue protective activity modulation. Protease cleavage sites may be included in the molecule.

A molecule may be conjugated to fully carbamylated erythropoietin of the invention through a polyfunctional molecule, i.e., a polyfunctional crosslinker. As used herein, the term “polyfunctional molecule” encompasses molecules having one functional group that can react more than one time in succession, such as formaldehyde, as well as molecules with more than one reactive group. As used herein, the term “reactive group” refers to a functional group on the crosslinker that reacts with a functional group on a molecule (e.g., peptide, protein, carbohydrate, nucleic acid, particularly a hormone, antibiotic, or anti-cancer agent to be delivered across an endothelial cell barrier) so as to form a covalent bond between the cross-linker and that molecule. The term “functional group” retains its standard meaning in organic chemistry. The polyfunctional molecules that can be used are preferably biocompatible linkers, i.e., they are noncarcinogenic, nontoxic, and substantially non-immunogenic in vivo. Polyfunctional cross-linkers such as those known in the art and described herein can be readily tested in animal models to determine their biocompatibility. The polyfunctional molecule is preferably bifunctional. As used herein, the term “bifunctional molecule” refers to a molecule with two reactive groups. The bifunctional molecule may be heterobifunctional or homobifunctional. A heterobifunctional cross-linker allows for vectorial conjugation. It is particularly preferred for the polyfunctional molecule to be sufficiently soluble in water for the cross-linking reactions to occur in aqueous solutions such as in aqueous solutions buffered at pH 6 to 8, and for the resulting conjugate to remain water soluble for more effective bio-distribution. Typically, the polyfunctional molecule covalently bonds with an amino or a sulfhydryl functional group. However, polyfunctional molecules reactive with other functional groups, such as carboxylic acids or hydroxyl groups, are contemplated in the present invention.

The homobifunctional molecules have at least two reactive functional groups, which are the same. The reactive functional groups on a homobifunctional molecule include, for example, aldehyde groups and active ester groups. Homobifunctional molecules having aldehyde groups include, for example, glutaraldehyde and subaraldehyde. The use of glutaraldehyde as a cross-linking agent was disclosed by Poznansky et al., Science 223, 1304-1306 (1984). Homobifunctional molecules having at least two active ester units include esters of dicarboxylic acids and N-hydroxysuccinimide. Some examples of such N-succinimidyl esters include disuccinimidyl suberate and dithio-bis-(succinimidyl propionate), and their soluble bis-sulfonic acid and bis-sulfonate salts such as their sodium and potassium salts. These homobifunctional reagents are available from Pierce, Rockford, Ill.

The heterobifunctional molecules have at least two different reactive groups. The reactive groups react with different functional groups, e.g., present on the carbamylated erythropoietin and the molecule. These two different functional groups that react with the reactive group on the heterobifunctional cross-linker are usually an amino group, e.g., a sulfhydryl group, e.g., the thiol group of cysteine; a carboxylic acid, e.g., the carboxylate on aspartic acid; or a hydroxyl group, e.g., the hydroxyl group on serine.

Of course, the carbamylated erythropoietin, may not have suitable reactive groups available for use with certain cross-linking agent; however, one of skill in the art will be amply aware of the choice of cross-linking agents based on the available groups for cross-linking in the fully carbamylated erythropoietin of the invention.

When a reactive group of a heterobifunctional molecule forms a covalent bond with an amino group, the covalent bond will usually be an amido or imido bond. The reactive group that forms a covalent bond with an amino group may, for example, be an activated carboxylate group, a halocarbonyl group, or an ester group. The preferred halocarbonyl group is a chlorocarbonyl group. The ester groups are preferably reactive ester groups such as, for example, an N-hydroxy-succinimide ester group.

The other functional group typically is either a thiol group, a group capable of being converted into a thiol group, or a group that forms a covalent bond with a thiol group. The covalent bond will usually be a thioether bond or a disulfide. The reactive group that forms a covalent bond with a thiol group may, for example, be a double bond that reacts with thiol groups or an activated disulfide. A reactive group containing a double bond capable of reacting with a thiol group is the maleimido group, although others, such as acrylonitrile, are also possible. A reactive disulfide group may, for example, be a 2-pyridyldithio group or a 5,5′-dithio-bis-(2-nitrobenzoic acid) group. Some examples of heterobifunctional reagents containing reactive disulfide bonds include N-succinimidyl 3-(2-pyridyl-dithio) propionate (Carlsson, et al., 1978, Biochem J., 173:723-737), sodium S-4-succinimidyloxycarbonyl-alpha-methylbenzylthiosulfate, and 4-succinimidyloxycarbonyl-alpha-methyl-(2-pyridyldithio)toluene. N-succinimidyl 3-(2-pyridyldithio)propionate is preferred. Some examples of heterobifunctional reagents comprising reactive groups having a double bond that reacts with a thiol group include succinimidyl 4-(N-maleimidomethyl)cyclohexane-1-carboxylate and succinimidyl m-maleimidobenzoate.

Other heterobifunctional molecules include succinimidyl 3-(maleimido)propionate, sulfosuccinimidyl 4-(p-maleimido-phenyl)butyrate, sulfosuccinimidyl 4-(N-maleimidomethyl-cyclohexane)-1-carboxylate, maleimidobenzoyl-N-hydroxy-succinimide ester. The sodium sulfonate salt of succinimidyl m-maleimidobenzoate is preferred. Many of the above-mentioned heterobifunctional reagents and their sulfonate salts are available from Pierce Chemical Co., Rockford, Ill. USA.

The need for the above-described conjugated to be reversible or labile may be readily determined by the skilled artisan. A conjugate may be tested in vitro for both the tissue protective activity, and for the desirable pharmacological activity. If the conjugate retains both properties, its suitability may then be tested in vivo. If the conjugated molecule requires separation from carbamylated erythropoietin for activity, a labile bond or reversible association with carbamylated erythropoietin will be preferable. The lability characteristics may also be tested using standard in vitro procedures before in vivo testing.

Additional information regarding how to make and use these as well as other polyfunctional reagents may be obtained from the following publications or others available in the art:
Carlsson, J. et al., 1978, Biochem. J. 173:723-737.
Cumber, J. A. et al., 1985, Methods in Enzymology 112:207-224.
Jue, R. et al., 1978, Biochem 17:5399-5405.
Sun, T. T. et al., 1974, Biochem. 13:2334-2340.
Blattler, W. A. et al., 1985, Biochem. 24:1517-152.
Liu, F. T. et al., 1979, Biochem. 18:690-697.
Youle, R. J. and Neville, D. M. Jr., 1980, Proc. Natl. Acad. Sci. U.S.A. 77:5483-5486.
Lerner, R. A. et al., 1981, Proc. Natl. Acad. Sci. U.S.A. 78:3403-3407.
Jung, S. M. and Moroi, M., 1983, Biochem. Biophys. Acta 761:162.
Caulfield, M. P. et al., 1984, Biochem. 81:7772-7776.
Staros, J. V., 1982, Biochem. 21:3950-3955.
Yoshitake, S. et al., 1979, Eur. J. Biochem. 101:395-399.
Yoshitake, S. et al., 1982, J. Biochem. 92:1413-1424.
Pilch, P. F. and Czech, M. P., 1979, J. Biol. Chem. 254:3375-3381.
Novick, D. et al., 1987, J. Biol. Chem. 262:8483-8487.
Lomant, A. J. and Fairbanks, G., 1976, J. Mol. Biol. 104:243-261.
Hamada, H. and Tsuruo, T., 1987, Anal. Biochem. 160:483-488.
Hashida, S. et al., 1984, J. Applied Biochem. 6:56-63.

Additionally, methods of cross-liking are reviewed by Means and Feeney, 1990, Bioconjugate Chem. 1:2-12.

Barriers which are crossed by the above-described methods and compositions of the present invention include but are not limited to the blood-brain barrier, the blood-eye barrier, the blood-testes barrier, the blood-ovary barrier, and the blood-uterus barrier.

Candidate molecules for transport across an endothelial cell barrier include, for example, hormones, such as growth hormone, neurotrophic factors, antibiotics, antivirals, or antifungals such as those normally excluded from the brain and other barriered organs, peptide radiopharmaceuticals, antisense drugs, antibodies and antivirals against biologically-active agents, pharmaceuticals, and anti-cancer agents. Non-limiting examples of such molecules include hormones such as growth hormone, nerve growth factor (NGF), brain-derived neurotrophic factor (BDNF), ciliary neurotrophic factor (CNTF), basic fibroblast growth factor (bFGF), transforming growth factor β1(TGFβ1), transforming growth factor β2(TGFβ2), transforming growth factor β3 (TGFβ3), interleukin 1, interleukin 2, interleukin 3, and interleukin 6, AZT, antibodies against tumor necrosis factor, and immunosuppressive agents such as cyclosporin. Additionally, dyes or markers may be attached to erythropoietin or one of the tissue protective cytokines of the present invention in order to visualize cells, tissues, or organs within the brain and other barriered organs for diagnostic purposes. As an example, a marker used to visualize plaque within the brain could be attached to erythropoietin or a tissue protective cytokine in order to determine the progression of Alzheimer's disease within a patient.

The present invention is also directed to a composition comprising a molecule to be transported via transcytosis across an endothelial cell tight junction barrier and a carbamylated erythropoietin as described above. The invention is for further directed to the use of a conjugate between a molecule and a carbamylated erythropoietin as described above for the preparation of a pharmaceutical composition for the delivery of the molecule across a barrier as described above.

### Pharmaceutical Composition

The tissue protective activity of carbamylated erythropoietin has been noted in PCT applications PCT/US01/49479, PCT/US03/20964, PCT/US03/21350, PCT/US04/15733, and PCT/US04/15863, and U.S. application Ser. No. 10/185,841, all incorporated by reference herein. Generally, the carbamylated erythropoietins resulting from the current method are useful for the therapeutic or prophylactic treatment of human diseases of the central nervous system or peripheral nervous system which have primarily neurological or psychiatric symptoms, ophthalmic diseases, cardiovascular diseases, cardiopulmonary diseases, respiratory diseases, kidney, urinary and reproductive diseases, bone diseases, skin diseases, gastrointestinal diseases and endocrine and metabolic abnormalities. In particular, such conditions and diseases include hypoxic conditions, which adversely affect excitable tissues, i.e. tissues responsive to the tissue protective effects of carbamylated erythropoietin as disclosed within PCT/US03/20984 and U.S. patent application Ser. No. 10/185,841, including, but not limited to such excitable tissues as the central nervous system tissue, peripheral nervous system tissue, or cardiac tissue or retinal tissue or renal tissue such as, for example, brain, heart, retina/eye, or kidney.

Therefore, the pharmaceutical compositions of the current invention can be used to treat or prevent damage to excitable tissue resulting from hypoxic conditions in a variety of conditions and circumstances including but not limited to retinal ischemia, macular degeneration, retinal detachment, retinitis pigmentosa, arteriosclerotic retinopathy, hypertensive retinopathy, retinal artery blockage, retinal vein blockage, hypotension, diabetic retinopathy, treatment of neurotoxin poisoning (such as domoic acid shellfish poisoning, neurolathyrism, and Guam disease, amyotrophic lateral sclerosis, and Parkinson's disease), mood disorders, anxiety disorders, depression, autism, attention deficit hyperactivity disorder, cognitive dysfunction, sleep disruption (for example, sleep apnea and travel-related disorders), subarachnoid and aneurismal bleeds, hypotensive shock, concussive injury, septic shock, anaphylactic shock, and sequelae of various encephalitides and meningitides (for example, connective tissue disease-related cerebritides such as lupus), postoperative treatment for embolic or ischemic injury; whole brain irradiation, sickle cell crisis, eclampsia, treatment of inhalation poisoning (such as carbon monoxide and smoke inhalation), severe asthma, adult respiratory distress syndrome, choking and near drowning, include hypoglycemia that may occur in inappropriate dosing of insulin, or with insulin-producing neoplasms (insulinoma), mitochondrial dysfunction, age-related loss of cognitive function and senile dementia, chronic seizure disorders, Alzheimer's disease, Parkinson's disease, dementia, memory loss, amyotrophic lateral sclerosis, multiple sclerosis, tuberous sclerosis, Wilson's Disease, cerebral and progressive supranuclear palsy, Guam disease, Lewy body dementia, prion diseases (such as spongiform encephalopathies, e.g., Creutzfeldt-Jakob disease), Huntington's disease, myotonic dystrophy, Freidrich's ataxia and other ataxias, Gilles de la Tourette's syndrome, seizure disorders (such as epilepsy and chronic seizure disorder), stroke, brain or spinal cord trauma, AIDS dementia, alcoholism, autism, retinal ischemia, glaucoma, autonomic function disorders (such as hypertension and sleep disorders), neuropsychiatric disorders (such as schizophrenia, schizoaffective disorder, attention deficit disorder, dysthymic disorder, major depressive disorder, mania, obsessive-compulsive disorder, psychoactive substance use disorders, anxiety, panic disorder, as well as unipolar and bipolar affective disorders), neuropathies (such as diabetic neuropathy or chemotherapy induced neuropathy), sepsis, and wound healing (including bed sores). Non-limiting examples of such conditions and circumstances are provided in the table herein below.

| Cell, tissue or | Dysfunction or | | |
|---|---|---|---|
| organ | pathology | Condition or disease | Type |
| Heart | Ischemia | Coronary artery | Acute, chronic |
| | | disease | Stable, unstable |
| | | Myocardial | Dressier's syndrome |
| | | infarction | |
| | | Angina | |
| | | Congenital heart | Valvular |
| | | disease | Cardiomyopathy |
| | | Prinzmetal angina | |
| | | Cardiac rupture | Aneurysmatic |
| | | | Septal perforation |
| | | Angiitis | |
| | Arrhythmia | Tachy-, | Stable, unstable |
| | | bradyarrhythmia | Hypersensitive carotid sinus |
| | | Supraventricular, | node |
| | | ventricular | |
| | | Conduction | |
| | | abnormalities | |
| | Congestive heart | Left, right, bi- | Cardiomyopathies, such as |
| | failure | ventricular | idiopathic familial, infective, |
| | | | metabolic, storage disease, |
| | | | deficiencies, connective tissue |
| | | | disorder, infiltration and |
| | | | granulomas, neurovascular |
| | | Myocarditis | Autoimmune, infective, |
| | | | idiopathic |
| | | Cor pulmonale | |
| | Blunt and penetrating | | |
| | trauma | | |
| | Toxins | Cocaine | |
| Vascular | Hypertension | Primary, secondary | |
| | Decompression | | |
| | sickness | | |
| | Fibromuscular | | |
| | hyperplasia | | |
| | Aneurysm | Dissecting, ruptured, | |
| | | enlarging | |
| Lungs | Obstructive | Asthma | |
| | | Chronic bronchitis, | |
| | | Emphysema and | |
| | | airway obstruction | |
| | Ischemic lung disease | Pulmonary | |
| | | embolism, | |
| | | Pulmonary | |
| | | thrombosis, | |
| | | Fat embolism | |
| | Environmental lung | | |
| | diseases | | |
| | Ischemic lung disease | Pulmonary embolism | |
| | | Pulmonary | |
| | | thrombosis | |
| | Interstitial lung | Idiopathic pulmonary | |
| | disease | fibrosis | |
| | Congenital | Cystic fibrosis | |
| | Cor pulmonale | | |
| | Trauma | | |
| | Pneumonia and | Infectious, parasitic, | |
| | pneumonitides | toxic, traumatic, | |
| | | burn, aspiration | |
| | Sarcoidosis | | |
| Pancreas | Endocrine | Diabetes mellitus, | Beta cell failure, dysfunction |
| | | type I and II | Diabetic neuropathy |
| | | Other endocrine cell | |
| | | failure of the | |
| | | pancreas | |
| | Exocrine | Exocrine pancreas | Pancreatitis |
| | | failure | |
| Bone | Osteopenia | Primary | Hypogonadism |
| | | secondary | immobilisation |
| | | | Postmenopausal |
| | | | Age-related |
| | | | Hyperparathyroidism |
| | | | Hyperthyroidism |
| | | | Calcium, magnesium, |
| | | | phosphorus and/or vitamin D |
| | | | deficiency |
| | Osteomyelitis | | |
| | Avascular necrosis | | |
| | Trauma | | |
| | Paget's disease | | |
| Skin | Alopecia | Areata | Primary |
| | | Totalis | Secondary |
| | | | Male pattern baldness |
| | Vitiligo | Localized | Primary |
| | | Generalized | Secondary |
| | Diabetic ulceration | | |
| | Peripheral vascular | | |
| | disease | | |
| | Burn injuries | | |
| Autoimmune | Lupus | | |
| disorders | erythematodes, | | |
| | Sjögren's syndrome, | | |
| | Rheumatoid arthritis, | | |
| | Glomerulonephritis, | | |
| | Angiitis | | |
| | Langerhan's | | |
| | histiocytosis | | |
| Eye | Optic neuritis | | |
| | Blunt and penetrating | | |
| | injuries, Infections, | | |
| | Sarcoid, Sickle Cell | | |
| | disease, Retinal | | |
| | detachment, | | |
| | Temporal arteritis | | |
| | Retinal ischemia, | | |
| | macular | | |
| | degeneration, retinal | | |
| | detachment, retinitis | | |
| | pigmentosa, | | |
| | arteriosclerotic | | |
| | retinopathy, | | |
| | hypertensive | | |
| | retinopathy, retinal | | |
| | artery blockage, | | |
| | retinal vein blockage, | | |
| | hypotension, and | | |
| | diabetic retinopathy. | | |
| Embryonic and | Asphyxia | | |
| fetal disorders | Ischemia | | |
| CNS | Chronic fatigue | | |
| | syndrome, acute and | | |
| | chronic hypoosmolar | | |
| | and hyperosmolar | | |
| | syndromes, AIDS | | |
| | Dementia, | | |
| | Electrocution | | |
| | Encephalitis | Rabies, Herpes | |
| | Meningitis | | |
| | Subdural hematoma | | |
| | Nicotine addiction | | |
| | Drug abuse and | Cocaine, heroin, | |
| | withdrawal | crack, marijuana, | |
| | | LSD, PCP, poly-drug | |
| | | abuse, ecstasy, | |
| | | opioids, sedative | |
| | | hypnotics, | |
| | | amphetamines, | |
| | | caffeine | |
| | Obsessive- | | |
| | compulsive disorders | | |
| | Spinal stenosis, | | |
| | Transverse myelitis, | | |
| | Guillian Barre, | | |
| | Trauma, Nerve root | | |
| | compression, | | |
| | Tumoral | | |
| | compression, Heat | | |
| | stroke | | |
| ENT | Tinnitus | | |
| | Meuniere's syndrome | | |
| | Hearing loss | | |
| | Traumatic injury, | | |
| | barotrauma | | |
| Kidney | Renal failure | Acute, chronic | Vascular/ischemic, interstitial |
| | | | disease, diabetic kidney |
| | | | disease, nephrotic syndromes, |
| | | | infections |
| | Henoch Schönlein | | |
| | Purpura | | |
| Striated muscle | Autoimmune | Myasthenia gravis | |
| | disorders | Dermatomyositis | |
| | | Polymyositis | |
| | Myopathies | Inherited metabolic, | |
| | | endocrine and toxic | |
| | Heat stroke | | |
| | Crush injury | | |
| | Rhabdomylosis | | |
| | Mitochondrial | | |
| | disease | | |
| | Infection | Necrotizing fasciitis | |
| Sexual | Central and | Impotence secondary | |
| dysfunction | peripheral | to medication | |
| Liver | Hepatitis | Viral, bacterial, | |
| | | parasitic | |
| | Ischemic disease | | |
| | Cirrhosis, fatty liver | | |
| | Infiltrative/metabolic | | |
| | diseases | | |
| Gastrointestinal | Ischemic bowel | | |
| | disease | | |
| | Inflammatory bowel | | |
| | disease | | |
| | Necrotizing | | |
| | enterocolitis | | |
| Organ | Treatment of donor | | |
| transplantation | and recipient | | |
| Reproductive | Infertility | Vascular | |
| tract | | Autoimmune | |
| | | Uterine abnormalities | |
| | | Implantation | |
| | | disorders | |
| Endocrine | Glandular hyper- and | | |
| | hypofunction | | |

One of ordinary skill in the art would understand that the pharmaceutical composition of the present invention may be made of a mixture of the carbamylated erythropoietins of the present invention as well as other therapeutics, including, but not limited to other tissue protective cytokines.

In one embodiment, such a pharmaceutical composition of carbamylated erythropoietin may be administered systemically to protect or enhance the target cells, tissue or organ. Such administration may be parenterally, via inhalation, or transmucosally, e.g., orally, nasally, rectally, intravaginally, sublingually, submucosally or transdermally. Preferably, administration is parenteral, e.g., via intravenous or intraperitoneal injection, and also including, but is not limited to, intra-arterial, intramuscular, intradermal and subcutaneous administration.

For other routes of administration, such as by use of a perfusate, injection into an organ, or other local administration, a pharmaceutical composition will be provided which results in similar levels of a tissue protective cytokine as described above. A level of about 15 pM-30 nM is preferred.

The pharmaceutical compositions of the invention may comprise a therapeutically effective amount of carbamylated erythropoietin, and a pharmaceutically acceptable carrier. Preferably, the therapeutically effective amount of carbamylated erythropoietin is non-toxic. In a specific embodiment, the term “pharmaceutically acceptable” means approved by a regulatory agency of the Federal or a state government or listed in the U.S. Pharmacopeia or other generally recognized foreign pharmacopeia for use in animals, and more particularly in humans. The term “carrier” refers to a diluent, adjuvant, excipient, or vehicle with which the therapeutic is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

Pharmaceutical compositions adapted for oral administration may be provided as capsules or tablets; as powders or granules; as solutions, syrups or suspensions (in aqueous or non-aqueous liquids); as edible foams or whips; or as emulsions. Tablets or hard gelatine capsules may comprise lactose, starch or derivatives thereof, magnesium stearate, sodium saccharine, cellulose, magnesium carbonate, stearic acid or salts thereof. Soft gelatine capsules may comprise vegetable oils, waxes, tats, semi-solid, or liquid polyols etc. Solutions and syrups may comprise water, polyols and sugars.

An active agent intended for oral administration may be coated with or admixed with a material that delays disintegration and/or absorption of the active agent in the gastrointestinal tract (e.g., glyceryl monostearate or glyceryl distearate may be used). Thus, the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions.

Pharmaceutical compositions adapted for transdermal administration may be provided as discrete patches intended to remain in intimate contact with the epidermis of the recipient for a prolonged period of time. Pharmaceutical compositions adapted for topical administration may be provided as ointments, creams, suspensions, lotions, powders, solutions, pastes, gels, sprays, aerosols or oils. For topical administration to the skin, mouth, eye or other external tissues a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base. Pharmaceutical compositions adapted for topical administration to the eye include eye drops. In these compositions, the active ingredient can be dissolved or suspended in a suitable carrier, e.g., in an aqueous solvent. Pharmaceutical compositions adapted for topical administration in the mouth include lozenges, pastilles and mouthwashes.

Pharmaceutical compositions adapted for nasal and pulmonary administration may comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, compositions adopted for nasal administration may comprise liquid carriers, e.g., nasal sprays or nasal drops. Alternatively, inhalation of compounds directly into the lungs may be accomplished by inhalation deeply or installation through a mouthpiece into the oropharynx. These compositions may comprise aqueous or oil solutions of the active ingredient. Compositions for administration by inhalation may be supplied in specially adapted devices including, but not limited to, pressurized aerosols, nebulizers or insufflators, which can be constructed so as to provide predetermined dosages of the active ingredient. In a preferred embodiment, pharmaceutical compositions of the invention are administered into the nasal cavity directly or into the lungs via the nasal cavity or oropharynx.

Pharmaceutical compositions adapted for rectal administration may be provided as suppositories or enemas. Pharmaceutical compositions adapted for vaginal administration may be provided as pessaries, tampons, creams, gels, pastes, foams or spray formulations.

Pharmaceutical compositions adapted for parenteral administration include aqueous and non-aqueous sterile injectable solutions or suspensions, which may contain antioxidants, buffers, bacteriostats and solutes that render the compositions substantially isotonic with the blood of an intended recipient. Other components that may be present in such compositions include water, alcohols, polyols, glycerine and vegetable oils, for example. Compositions adapted for parenteral administration may be presented in unit-dose or multi-dose containers, for example sealed ampules and vials, and may be stored in a freeze-dried (lyophilized) condition requiring only the addition of a sterile liquid carrier, e.g., sterile saline solution for injections, immediately prior to use. Extemporaneous injection solutions and suspensions may be prepared from sterile powders, granules and tablets. In one embodiment, an autoinjector comprising an injectable solution of carbamylated erythropoietin may be provided for emergency use by ambulances, emergency rooms, and battlefield situations, and even for self-administration in a domestic setting, particularly where the possibility of traumatic amputation may occur, such as by imprudent use of a lawn mower.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically-sealed container such as an ampule or sachette indicating the quantity of active agent. Where the composition is to be administered by infusion, it can be dispensed with an infusion bottle containing sterile pharmaceutical grade water or saline. Where the composition is administered by injection, an ampule of sterile saline can be provided so that the ingredients may be mixed prior to administration.

Suppositories generally contain active ingredient in the range of 0.5% to 10% by weight; oral formulations preferably contain 10% to 95% active ingredient.

A perfusate composition may be provided for use in transplanted organ baths, for in situ perfusion, or for administration to the vasculature of an organ donor prior to organ harvesting. Such pharmaceutical compositions may comprise levels of carbamylated erythropoietin not suitable for acute or chronic, local or systemic administration to an individual, but will serve the functions intended herein in a cadaver, organ bath, organ perfusate, or in situ perfusate prior to removing or reducing the levels of the carbamylated erythropoietin contained therein before exposing or returning the treated organ or tissue to regular circulation.

The present invention provides pharmaceutical compositions for the treatment, prophylaxis, and amelioration of one or more symptoms associated with hypoxia, ischemia, trauma, and/or inflammation. In a specific embodiment, a composition comprises carbamylated erythropoietin or carbamylated erythropoietin and another tissue protective cytokine. In another embodiment, a composition comprises carbamylated erythropoietin or carbamylated erythropoietin and one or more tissue protective cytokines, and one or more prophylactic or therapeutic agents other than tissue protective cytokines, said prophylactic or therapeutic agents known to be useful for, or having been or currently being used in the prevention, treatment or amelioration of one or more symptoms associated inflammation, hypoxia, ischemia, or trauma.

In a preferred embodiment, a composition of the invention is a pharmaceutical composition. Such compositions comprise a prophylactically or therapeutically effective amount of one or more prophylactic or therapeutic agents (e.g., a tissue protective cytokine or other prophylactic or therapeutic agent), and a pharmaceutically acceptable carrier. In one embodiment, the term “therapeutically effective amount” means including an amount of an agent that is not necessarily effective when the agent is administered alone but is effective when co-administered with another agent. Therapeutically effective amounts of carbamylated erythropoietin of the current invention include 1 pg to 5 mg, 500 pg to 5 mg, 1 ng to 5 mg, 500 ng to 5 mg, 1 μg to 5 mg, 500 μg to 5 mg, or 1 mg to 5 mg of a tissue protective cytokine, and a pharmaceutically acceptable carrier. In a preferred embodiment, the amount of tissue protective cytokine is within the range from about 1 pg to 1 mg.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In particular, the invention provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted, e.g., with water or saline to the appropriate concentration for administration to a subject. Preferably, one or more of the prophylactic or therapeutic agents, or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents, or pharmaceutical compositions of the invention should be stored at between 2 and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents, or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Generally, the ingredients of the compositions of the invention are derived from a subject that is the same species origin or species reactivity as recipient of such compositions.

In another embodiment, for example, the carbamylated erythropoietin can be delivered in a controlled-release system. For example, the polypeptide may be administered using intravenous infusion, an implantable osmotic pump, a transdermal patch, liposomes, or other modes of administration. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, the compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); WO 91/04014; U.S. Pat. No. 4,704,355; Lopez-Berestein, ibid., pp. 317-327; see generally ibid.). In another embodiment, polymeric materials can be used (see Medical Applications of Controlled Release, Langer and Wise (eds.), CRC Press: Boca Raton, Fla., 1974; Controlled Drug Bioavailability, Drug Product Design and Performance, Smolen and Ball (eds.), Wiley: N.Y. (1984); Ranger and Peppas, J. Macromol. Sci. Rev. Macromol. Chem. 23:61, 1953; see also Levy et al., 1985, Science 228:190; During et al., 1989, Ann. Neurol. 25:351; Howard et al., 1989, J. Neurosurg. 71:105).

In yet another embodiment, a controlled release system can be placed in proximity of the therapeutic target, i.e., the target cells, tissue or organ, thus requiring only a fraction of the systemic dose (see, e.g., Goodson, pp. 115-138 in Medical Applications of Controlled Release, vol. 2, supra, 1984). Other controlled release systems are discussed in the review by Langer (1990, Science 249:1527-1533).

In another embodiment, a carbamylated erythropoietin, as properly formulated, can be administered by nasal, oral, rectal, vaginal, or sublingual administration.

In a specific embodiment, it may be desirable to administer carbamylated erythropoietin of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as silastic membranes, or fibers.

Selection of the preferred effective dose will be readily determinable by a skilled artisan based upon considering several factors, which will be known to one of ordinary skill in the art. Such factors include the particular form of erythropoietin or the tissue protective cytokine, and its pharmacokinetic parameters such as bioavailability, metabolism, half-life, etc., which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether administration is acute or chronic, concomitant medications, and other factors well known to affect the efficacy of administered pharmaceutical agents. Thus the precise dosage should be decided according to the judgment of the practitioner and each patient's circumstances, e.g., depending upon the condition and the immune status of the individual patient, and according to standard clinical techniques.

In another aspect of the invention, a perfusate or perfusion solution is provided for perfusion and storage of organs for transplant, the perfusion solution includes an amount of carbamylated erythropoietin effective to protect responsive cells and associated cells, tissues or organs. Transplant includes but is not limited to xenotransplantation, where an organ (including cells, tissue or other bodily part) is harvested from one donor and transplanted into a different recipient; and autotransplant, where the organ is taken from one part of a body and replaced at another, including bench surgical procedures, in which an organ may be removed, and while ex vivo, resected, repaired, or otherwise manipulated, such as for tumor removal, and then returned to the original location. In one embodiment, the perfusion solution is the University of Wisconsin (UW) solution (U.S. Pat. No. 4,798,824) which contains from about 1 to about 25 U/ml carbamylated erythropoietin, 5% hydroxyethyl starch (having a molecular weight of from about 200,000 to about 300,000 and substantially free of ethylene glycol, ethylene chlorohydrin, sodium chloride and acetone); 25 mM KH₂PO₄; 3 mM glutathione; 5 mM adenosine; 10 mM glucose; 10 mM HEPES buffer; 5 mM magnesium gluconate; 1.5 mM CaCl₂; 105 mM sodium gluconate; 200,000 units/ml penicillin; 40 units/ml insulin; 16 mg dexamethasone; 12 mg/ml Phenol Red; and has a pH of 7.4-7.5 and an osmolality of about 320 mOsm/l. The solution is used to maintain cadaveric kidneys and pancreases prior to transplant. Using the solution, preservation can be extended beyond the 30-hour limit recommended for cadaveric kidney preservation. This particular perfusate is merely illustrative of a number of such solutions that can be adapted for the present use by inclusion of an effective amount of carbamylated erythropoietin. In a further embodiment, the perfusate solution contains from about 1 to about 500 ng/ml carbamylated erythropoietin, or from about 40 to about 320 ng/ml carbamylated erythropoietin. As mentioned above, any form of erythropoictin or tissue protective cytokines can be used in this aspect of the invention.

While the preferred recipient of a carbamylated erythropoietin for the purposes herein throughout is a human, the methods herein apply equally to other mammals, particularly domesticated animals, livestock, companion, and zoo animals. However, the invention is not so limiting and the benefits can be applied to any mammal.

In further aspects of the ex-vivo invention, carbamylated erythropoietin and any tissue protective cytokine such as but not limited to the ones described above may be employed.

In another aspect of the invention, methods and compositions for enhancing the viability of cells, tissues or organs which are not isolated from the vasculature by an endothelial cell barrier are provided by exposing the cells, tissue or organs directly to a pharmaceutical composition comprising carbamylated erythropoietin, or administering or contacting a pharmaceutical composition containing carbamylated erythropoietin to the vasculature of the tissue or organ. Enhanced activity of responsive cells in the treated tissue or organ is responsible for the positive effects exerted.

In the foregoing examples in which a carbamylated erythropoietin of the invention is used for ex-vivo applications, or to treat responsive cells such as neuronal tissue, retinal tissue, heart, lung, liver, kidney, small intestine, adrenal cortex, adrenal medulla, capillary endothelial, testes, ovary, or endometrial cells or tissue, the invention provides a pharmaceutical composition in dosage unit form adapted for protection or enhancement of responsive cells, tissues or organs distal to the vasculature.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLE #1

Erythropoietin was carbamylated according to the following procedure.

First, potassium cyanate (KOCN, MW 81.12) was recrystallized from water and ethanol. Next, a 1 M solution of sodium borate buffer having a pH of 8.7-9.2 was prepared from boric acid (H₃BO₃, MW 61.84) and sodium tetraborate, decahydrate (Na₂B₄O₇. 10H₂O, MW 381.4). The erythropoietin was concentrated using a Stirred Ultrafiltration Cell (Model 8200, Amicon) with an Ultrafiltration membrane filter (10,000 MW, filter code: PBCG, Amicon) to a concentration of 6 mg/ml (in this example, in a volume of 17.7 ml).

For carbamylation, the erythropoietin was first diluted with an equal volume of the 1M borate buffer in a 50 ml Plug Seal Cap tube. Next, a sufficient amount of recrystallized potassium cyanate was added to bring its concentration to 1M. The tube was then placed within an incubator set to a temperature of between 37.0 to 38.0° C. and incubated for 16 hours.

Immediately following carbamylation the erythropoietin was desalted by dialysis against 100 volumes of deionized water with multiple changes of the water. The dialyzed carbamylated erythropoietin was then purified using a Sephacryl S-100 column (HiPrep 26/60, Amersham-Pharmacia) with sodium phosphate buffer (50 mM, pH 7.2±0.1, with 0.15 M NaCl) attached to a AKTAprime system (Amersham-Pharmacia). The fractions pooled from the filtration column were concentrated using a centrifugal filter device, Amicon Ultra Centrifugal Filter Device (10,000 MWCO) in a Megafuge 1.0R centrifuge (Heraeus Instruments).

### Results of In Vitro Release Tests for the Carbamylated Erythropoietin

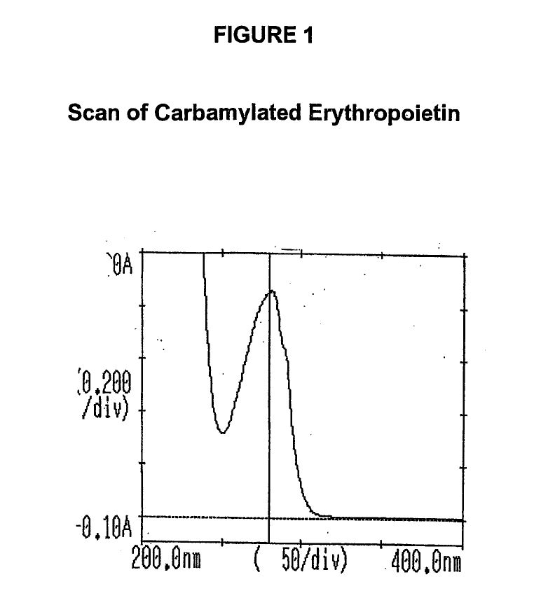

The carbamylated erythropoietin was then WV scanned using a UV-Visible Spectophotometer, Shimadzu UV-1601, to determine the protein content using A₂₈₀. The carbamylated erythropoietin had a maximum absorbance at 278-283 nm, minimum absorbance at 249-254 nm and no absorbance at >320 nm (FIG. 1). The A₂₈₀ was 0.853 for the 2 fold-diluted carbamylated erythropoietin. Based on the A₂₈₀ reading of diluted product, the protein content was calculated using the formula: (mg/ml)=(A₂₈₀×dilution fold)/0.743. The calculation result indicated that the protein content was 2.3 mg/ml.

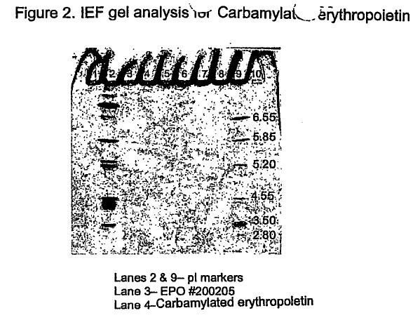

An IEF gel analysis was performed on the carbamylated erythropoietin. Samples of the carbamylated erythropoietin with 2× IEF sample buffer (pH 3-7, Novex Sample Buffer, LC5371) and IEF marker (Serva Liquid Mix IEF Marker 3-10, Invitrogen 39212-10) were loaded into an IEF gel (Novex IEF gel, EC6655B, Novex IEF Cathode Buffer, LC5370, Novex IEF Anode Buffer, LC5300). The gel was then run at 100V for 45 minutes, 200V for another 45 minutes, and 500 V for 15 minutes. Upon completion of the run, the gel was placed in fixing solution (12% (w/v) trichloroacetic acid and 3.5% 5-sulfosalicylic acid in water) for 10-15 minutes at room temperature on a rocking shaker. The gel was then rinsed 2-3 times with 100-150 ml of deionized water. The gel was then stained using 20-30 ml of staining solution (0.1% Coomassie Blue R-250, 50% methanol, and 10% acetic acid) for 5-6 minutes at room temperature, and was then rinsed with 20-30 ml of methanol-acetic acid (50% & 7%) solution 2-3 times. The gel was then destained 2-3 times in 50-100 ml of methanol-acetic acid (10%-7%) solution and then rinsed 2-3 times with 100-150 ml of deionized water. The gel was then dried using a DryErase Gel Drying System (Invitrogen). According to the IEF gel analysis (FIG. 2), the pI for the product was <3.5 and did not overlap with EPO, whose pI was in a range of 3.5-5 showing 6-7 isoform bands. This shows that the carbamylation was successfully performed.

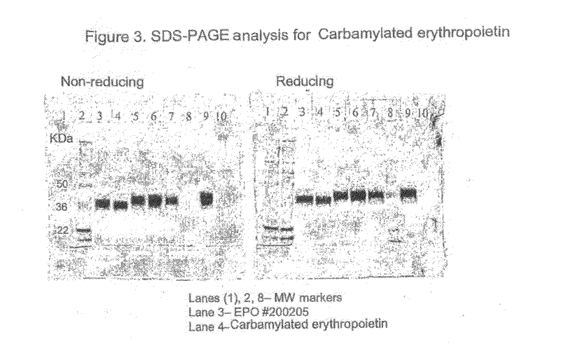

The carbamylated erythropoietin was then analyzed using SDS-PAGE under non-reducing and reducing conditions. For the non-reducing SDS-PAGE, a sample of the carbamylated erythropoietin mixed with 10 μl 2× tris-glycine SDS sample buffer (Invitrogen, LC2676) and heated at 85-95° C. for 3-5 minutes was loaded onto a Tris-Glycine gel (10%, Invitrogen, EC6075). The gel was run at 125 V for 90 minutes, at which time the gel was fixed in 100-200 ml of methanol-acetic acid solution (50% & 7%) for 15 minutes. The gel was then washed 4 times with 100-200 ml for minutes each time. The gel was then stained using 20-30 ml of GelCode Blue Stain Reagent Solution (Pierce) for at least 1 hr. with gentle shaking on a rocking shaker. The gel was then washed several times using deionized water until the background was cleared and then dried using the Dry Erase Gel Drying system. For reducing conditions the sample was mixed with 10 μl 2× tris-glycine SDS sample buffer containing 0.2 M DTT. As seen in the SDS-PAGE analysis under non-reducing and reducing conditions (FIG. 3), no obvious aggregate was detected for the product. A single band migrated at apparent MW ˜36 kDa for carbamylated erythropoietin, the same as its precursor erythropoietin.

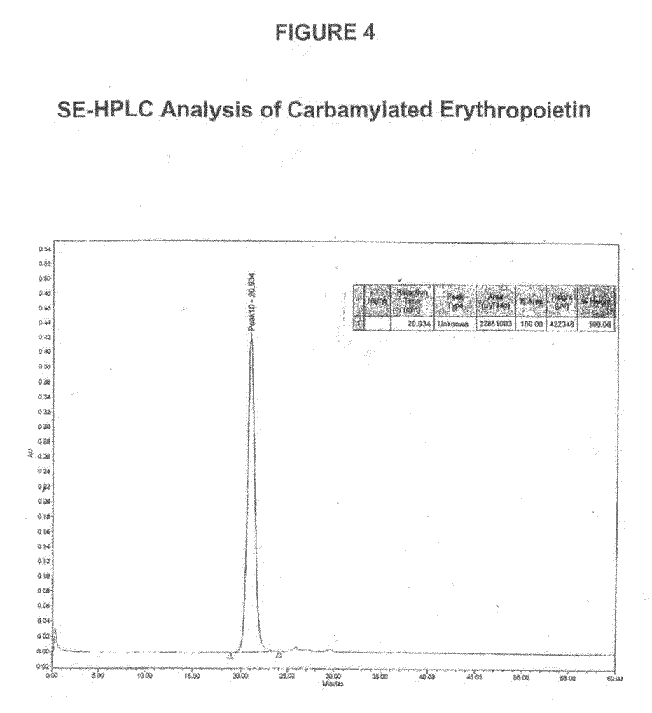

Next, SE-HPLC analysis was run on the carbamylated erythropoietin using a Waters 1525 Binary HPLC pump, Waters 2487 Dual Absorbance Detector, Waters 717 autosampler and Shodex GFC column (PROTEIN KW-803, 8.0 mm×300 mm). A sample of the carbamylated erythropoietin was diluted to 0.2 mg/ml with TSK buffer (8.1 1 mM Na₂HPO₄, 1.5 mM KH₂PO₄, 400 mM NaCl, pH 7.40±0.10) and run in the HPLC for 60 minutes, at a flow rate of 0.5 ml/min, with solvent at 100% TSK buffer, with a high pressure limit of 4000 PSI and lower limit of 0 PSI. The UV detectors settings were set to single wavelength and 2487 channel 1 absorbance enabled. The SEC-HPLC analysis (FIG. 4) confirmed that the protein purity was ˜100%, without detectable aggregate.

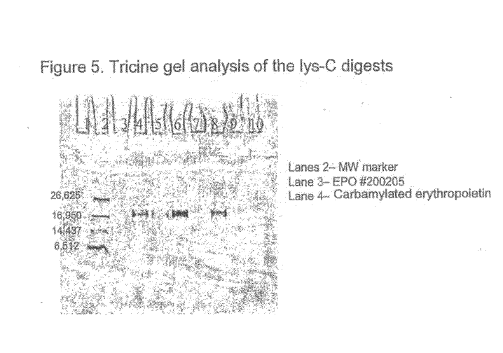

A sample of the carbamylated erythropoietin was then subjected to N-deglycosylation followed by Lys-C digestion to determine that all of the cleavable lysines were carbamylated. First, samples (50 μg) were mixed with 5 μl 1M NH₄HCO₃, 1 μl 0.1 M DTT, and 1 μl deionized water. The mixture was then heated to about 50-55° C. for 20 minutes, then kept at ambient temperature for another 15 to 20 minutes, after which, an additional 0.5 μl of 0.5 M IAA was added to the mixture and the mixture was incubated in the dark at ambient temperature for another 20 minutes. One (1) μl of PNGase F (N-glycosidase F, EC 3.5.1.52, MW 36 Kda, Calbiochem #362185) was added to the mixture and it was then incubated in a water bath at 37° C. for 18-24 hrs. The deglycosylation of the carbamylated erythropoietin was then confirmed using a 16% tricine gel (one major band at 17-27 Kda). Next, the deglycosylated carbamylated erythropoietin was subjected to Lys-C digestion by mixing 25 μl of the deglycosylated carbamylated erythropoietin with 0.5 μl of Lys-C(Lysyl Endopeptidase, EC 3.4.21.50, MW 27-30 Kda, Wako 125-02543) and incubated in a water bath at 37±0.5° for 18-24 hrs. The resulting product was then run on a 16% tricine gel. The tricine gel analysis of the Lys-C digests of deglycosylated products was shown in FIG. 5. EPO was digested into <6.5 KDa fragments due to the presence of unmodified lysine residues. But the carbamylated erythropoietin of the present invention was not digested, because it's cleavable lysine residues were completely carbamylated and thus resistant to the enzymatic digestion by Lys-C.

The carbamylated erythropoietin, erythropoietin and a blank were subjected to the TNBS assay noted above. The results of the assay are noted within the table below.

| | | | % Free | | |
|---|---|---|---|---|---|
| | | | Amino | Epo | |
| Compound | Peak At | Abs at Peak | Group | Activity | Figure |
| Erythropoietin | 347.5 nm | 1.5189 | 100 | Full | 9 |
| Blank | No Peak | A₃₄₀ = 0.2230 | 0 | N/A | 10 |
| Carbamylated | No Peak | A₃₄₆ = 0.2412 | 1.2 | ~0 | 11 |
| Erythropoietin | | | | | |

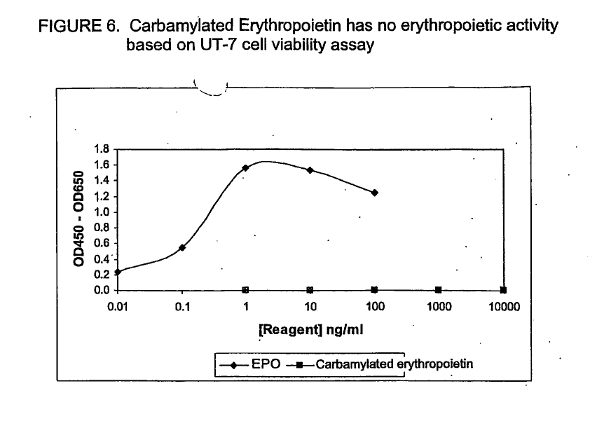

The carbamylated erythropoietin was tested for remaining erythropoietic activity by UT-7/EPOR cell viability assay in accordance with the procedure listed above. As seen in FIG. 6, no erythropoietic activity was detected at a concentration of 10 μg/ml for the carbamylated erythropoietin.

### Results of In Vivo Release Test for the Carbamylated Erythropoietin

The product was further tested for any tissue protective activity using a Sciatic Nerve Assay. Ten Sprague-Dawley rats (200-300 grams) (five per group—carbamylated erythropoietin treated group and PBS treated group) were used within the assay. The assay was performed by first anesthetizing the rat using isoflurane (Baxter NPC 10019-773-60) and Table Top Laboratory Anesthesia System (flowmeter set to 2-3 liters/minute @ 55 psi) for at least 3 minutes. The rat was then placed on a homeothermic blanket and a rectal probe was inserted to monitor the rat's core temperature to make sure that it was maintained at 35-37° C. during the operation. In order to assist with this the temperature of the operating room was maintained at least 23° C. Next, the right sciatic nerve was exposed at mid thigh through a quadriceps muscle dissection—a 2 cm incision with a 15 blade scalpel was made through the skin parallel and over the quadriceps muscle, using a pair of dissecting scissors the quadriceps muscle was cut to expose the sciatic nerve, and the nerve was freed from the surrounding membranes. A 2-0 braided silk thread (Ethicon, 685-G) was then passed under the nerve and the ends of the suture were tied and passed through a guide which was maintained perpendicular to the nerve. The end of the suture was then tied to a non-elastic cord which was then draped around the pulley system (a NYL pulley bearing MTD ¼″B (PO Number 04174-01) with stabilizer) and a 100 gram weight attached to the non-elastic cord was slowly released. The weight was allowed to hang for 1 minute before the silk suture was cut to release the weight. Using ½ cc insulin syringe a 10 μg/ml dose of the carbamylated erythropoietin or PBS was injected into the caudal vein and the muscle and surgical incision were closed, and 5 ml of Lactated Ringers solution was injected subcutaneously into the rat. The core temperature of the rat was maintained at 35-37° C. using a heat blanket during recovery.

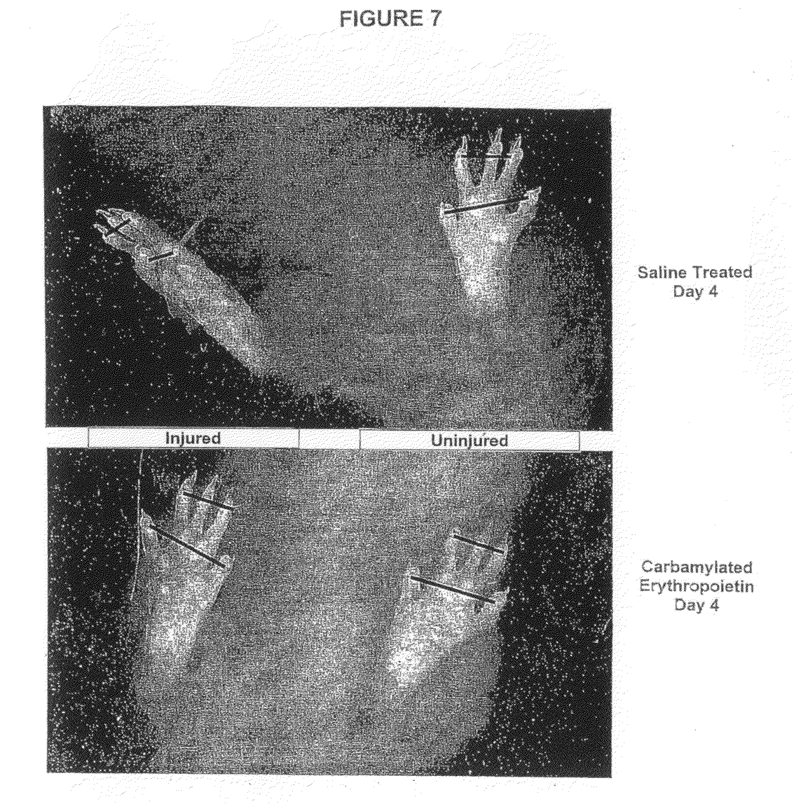

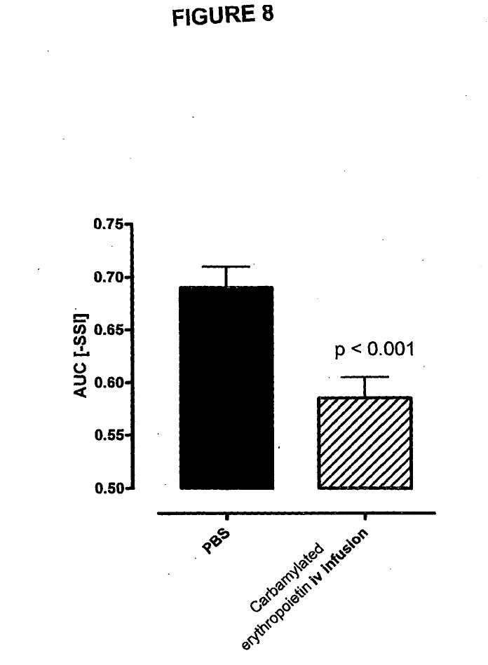

Over the next four days the rear toe splaying of the rats was determined by placing the rat in an acrylic tube with a diameter of 30 cm on the scanning surface of a digital scanner. After waiting 5 minutes in order to permit the rat to acclimate itself, a scan was taken of the rat's back feet that clearly displayed all 5 toes. Three acceptable scans of each rat were taken. From the scans the Toe-Spread, the difference between the ball of the first toe and the ball of the fifth toe, and Intermediate Toe Spread, the distance between the ball of the second toe and the bail of the fourth toe, were measured (FIG. 7). The static sciatic index was then computed in accordance with S. Erbayraktar et al., Proc Natl Acad Sci USA 100, 6741-6746 (2003) and statistical analysis was completed on the results. As can be seen in FIG. 8, the static sciatic index for the carbamylated erythropoietin was less than 0.65 and showed a significant improvement over the static sciatic nerve index (at 0.68) for the PBS treated rats.

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein are incorporated by reference herein in their entireties for all purposes.

What is claimed is:

1. A method for producing a carbamylated erythropoietin having less that about 10% free primary amines on the lysines and the N-terminal amino acids wherein the method comprises contacting an amount of erythropoietin at a concentration of less than 4 mg/ml, with a concentration of about 0.05 M to 2 M potassium cyanate, with a concentration of about 0.05 M to 0.5 M sodium borate buffer pH 7-10, at a temperature of about 30 to 38° C. for a period of about 1 to 24 hours wherein the carbamylated erythropoietin is not digested when exposed to Lys-C proteolysis, exhibits no erythropoietic activity in a TF-1 or UT-7/EPOR cell viability assay at a concentration of 1 μg/ml, and demonstrates a static sciatic index of less than about 0.65 within a Sciatic Nerve Assay.

2. The method of claim 1 wherein the carbamylated erythropoietin has less than about 7.5% free primary amines on the lysines and the N-terminal amino acids.

3. The method of claim 2, wherein the carbamylated erythropoietin has less than about 5% free primary amines on the lysines and the N-terminal amino acids.

5. The method of claim 1 wherein the concentration of erythropoietin is concentrated to about 1.1 mg/ml to about 2.5 mg/ml.

6. The method of claim 5 wherein the concentration of erythropoietin is about 2.2 mg/ml.

7. The method of claim 1 wherein the concentration of potassium cyanate is about 0.5 M to about 1.5 M.

8. The method of claim 7 wherein the concentration of potassium cyanate is about 1 M.

9. The method of claim 1 wherein the concentration of sodium borate buffer is about 0.1 M to about 0.5 M.

10. The method of claim 9 wherein the concentration of sodium borate buffer is about 0.5 M.

11. The method of claim 1 wherein the temperature is about 36° C. to about 38° C.

12. The method of claim 11 wherein the temperature is about 37° C.

13. The method of claim 1 wherein the period is about 14 to 24 hours.

14. The method of claim 13 wherein the period is about 16 hours.

15. The method of claim 1 wherein the carbamylated erythropoietin exhibits no erythropoietic activity in a TF-1 or UT-7/EPOR assay at a concentration of 10 μg/ml.

16. The method of claim 1 wherein the erythropoietin is recombinant erythropoietin, long acting erythropoietin, erythropoietin derivatives, erythropoietin analogs, erythropoietin conjugates, erythropoietin fusion proteins, chemically modified erythropoietin, erythropoietin muteins, expression-system-mediated glycosylation modifications of erythropoietin, synthetic erythropoietin, or naturally occurring erythropoietin.

17. The method of claim 16 wherein the erythropoietin is human erythropoietin.

18. The method of claim 18 wherein the erythropoietin is asialoerythropoietin.

19. The method of claim 1 wherein the static sciatic index is less than about 0.62.

20. The method of claim 19 wherein the static sciatic index is less than about 0.60.

21. A pharmaceutical composition comprising a non-toxic therapeutically effective amount of a carbamylated erythropoietin wherein the carbamylated erythropoietin has less than about 10% free primary amines on the lysines and the N-terminal amino acids is not digested when exposed to Lys-C proteolysis, exhibits no erythropoietic activity in a TF-1 or UT-7/EPOR cell viability assay at a concentration of 1 μg/ml, and demonstrates a static sciatic index of less than about 0.65 within a Sciatic Nerve Assay, and a pharmaceutically acceptable carrier.

22. The pharmaceutical composition of claim 21 wherein the carbamylated erythropoietin has less that about 7.5% free primary amines on the lysines and the N-terminal amino acids.

23. The pharmaceutical composition of claim 22 wherein the carbamylated erythropoietin has less that about 5% free primary amines on the lysines and the N-terminal amino acids.

24. The pharmaceutical composition of claim 21 wherein the carbamylated erythropoietin exhibits no erythropoietic activity in a TF-1 or UT-7/EPOR cell viability assay at a concentration of 10 μg/ml.

25. The pharmaceutical composition of claim 21 wherein the static sciatic index is less than 0.62

26. The pharmaceutical composition of claim 25 wherein the static sciatic index is less than 0.60.

27. A method for treating a condition or disease of an excitable tissue comprising administering a non-toxic amount of the pharmaceutical composition of claim 22.

28. A method of claim 27, wherein the excitable tissue is heart, eye or renal tissue.

29. A method of claim 27, wherein the condition or disease is optic neuritis, blunt or penetrating injuries to the eye, infections of the eye, sarcoid, sickle cell disease, retinal detachment, temporal arteritis, retinal ischemia, macular degeneration, retinal detachment, retinitis pigmentosa, arteriosclerotic retinopathy, hypertensive retinopathy, retinal artery blockage, retinal vein blockage, hypotension, diabetic retinopathy, diabetic neuropathy, coronary artery disease, myocardial infarction, Dressler's syndrome, angina, congenital heart disease, valvular cardiomyopathy, prinzmetal angina, cardiac rupture, aneurysmatic septal perforation, angiitis, arrhythmia, congestive heart failure, cardiomyopathies, myocarditis, cor pulmonale, blunt or penetrating traumas to the heart, toxic poisoning, renal failure, vascular/ischemic, interstitial disease, diabetic kidney disease, nephrotic syndromes, kidney infections, or Henoch Schönlein purpura.

30. The method of claim 1, wherein the carbamylated erythropoietin has less than 10% aggregates.

31. The method of claim 30, wherein the carbamylated erythropoietin has less than 6% aggregates.

32. The method of claim 31, wherein the carbamylated erythropoietin has less than 2% aggregates.
